(19) United States
(12) Patent Application Publication
(10) Pub. No.: US 2018/0015025 A1
(43) Pub. Date: Jan. 18, 2018

(54) Applicator-Free Deodorant

(71) Applicant: Luxy Duck LLC, North Andover, MA (US)

(72) Inventor: Gloria Hersom, North Andover, MA (US)

(21) Appl. No.: 15/213,038

(22) Filed: Jul. 18, 2016

(51) Int. Cl. A61K 8/92; A61K 8/19; A61K 8/02; A61Q 15/00; A61K 8/97

(52) U.S. Cl. CPC A61K 8/92; A61Q 15/00; A61K 8/97; A61K 8/022; A61K 8/0216; A61K 8/922; A61K 8/19; A61K 2800/24; A61K 2800/592

(57) ABSTRACT

An applicator-free deodorant formed into a three dimensional body that is easy to grasp and discretely place in the underarm in a single step where it melts by body heat, thereby coating the skin of the underarm and functioning as a deodorant is disclosed. In one embodiment, the applicator free deodorant is a cube, although other shapes are contemplated. The applicator-free deodorant includes a plant based oil and at least one absorbent powder, and may optionally include sodium bicarbonate, an essential oil, or naturally occurring wax.

### CROSS-REFERENCE TO RELATED APPLICATIONS

### Technical Field

This disclosure is related generally to an applicator-free deodorant, and more particularly to a deodorant made from natural ingredients that is formed into a solid, three dimensional body sized for single-use application, in which the three dimensional body is placed in the underarm of the user, where it melts by body heat and disperses across the skin of the underarm without use of a traditional applicator.

### Background

The deodorant and antiperspirant industry for underarm hygiene purposes was an $18 billion industry in the United States in 2015. A steady increase in the size of this market is predicted in both the United States and globally. A limited number of dispensing options exist for application of deodorant or antiperspirant by the user, and waste, in the form of disposable applicator materials, is a considerable environmental factor. Common methods for the application of deodorants and antiperspirants are via roll on applicators, stick deodorants contained within a plastic applicator, and aerosol sprays. All of these application methods require the use of an external applicator which provides a housing for supporting, dispensing and applying the deodorant or antiperspirant product.

Roll on and stick applicators are generally made of a plastic container, which supports a conventional deodorant or antiperspirant product, a cover, and in the case of a roll on applicator, a large plastic ball is also required for application of the product. These applicators generate a large amount of plastic waste, which is often non-recyclable. Aerosol cans are conventionally used for the application of spray deodorants and antiperspirants, and like plastic deodorant and antiperspirant containers, may not be recyclable. In each of these instances, the volume of packaging is large in relation to the volume of deodorant or antiperspirant products. Because this packaging may not be recyclable, and instead, disposed of in a landfill after the product is consumed, conventional deodorant and antiperspirant packaging generates a significant waste stream.

In addition to concerns about increased landfill waste, the ingredients in conventional deodorants and antiperspirants have also given rise to health and environmental concerns. Aluminum, certain antibacterial agents, such as triclosan, which is a common ingredient in conventional deodorants and antiperspirants, and parabens are just a few ingredients that have been linked to health concerns. The plastic packaging of conventional deodorants and antiperspirants is derived from petrochemicals, the production of which also gives rise to health and environmental concerns, including climate change. Finally, the volatile organic compounds used as propellants in deodorants and antiperspirants applied via an aerosol spray can contribute to an increase in ground level ozone and smog.

In addition to the health and environmental concerns arising from many conventional deodorants and antiperspirants, these products are not always easily portable due to the large size of the containers and applicators. For example, restrictions on the volume of products that may be taken aboard airplanes can make travelling with a conventional deodorant or antiperspirant product inconvenient. Conventional deodorants and antiperspirants are often provided in travel size containers, and single use applicators are also available and provide a convenient alternative for users who would like to freshen up during the day or while traveling. Unfortunately, these alternatives contribute to increased waste in the form of disposable applicators and packaging.

Patent Publ. No. US 2015/0030368 A1 describes a single-use, applicator for use in applying a deodorant, antiperspirant or other cosmetic. The applicator is approximately the size and shape of a credit card, with a deodorant or antiperspirant adhered to the applicator card. The card is used to apply the deodorant or antiperspirant to the underarm by rubbing the surface of the applicator card onto the skin of the user, as with other conventional deodorant application methods. Although smaller and more easily transportable than conventional deodorant or antiperspirant application methods, the applicator card and its packaging are single-use and disposable, thus contributing to increased waste.

Patent Publ. No. US 2015/0321028 A1 discloses a deodorant tablet that is contained in individual blister packaging with an individual compartment for each deodorant tablet. The deodorant tablet is encased in a protective, non-deodorant coating. When applied to the underarm, the coating material dissolves and evaporates, allowing the user to distribute the conventional deodorant or antiperspirant product encapsulated therein to the underarm. Because the tablet coating takes time to evaporate, the user must hold or rub the tablet in the underarm for some period of time in order to apply the deodorant material, which may be difficult or messy given of the small size of the tablet.

Patent Publ. No. US 2007/021,092 discloses an underarm hygiene product in which a deodorant patch is positioned on an application carrier sheet, which is used to apply deodorant or antiperspirant to the underarm. In order to apply the deodorant or antiperspirant to the underarm, the user must position the carrier sheet to the skin of the underarm. The application carrier sheet remains on the skin while the deodorant or antiperspirant product dissolves, thereby disassociating from the carrier sheet. The user must remove the carrier sheet from the underarm after the deodorant or antiperspirant product has disassociated form the sheet.

As will be appreciated, conventional deodorant and antiperspirant compositions and packaging can give rise to health and environmental concerns stemming from potentially harmful ingredients and disposable packaging. Certain known deodorant and antiperspirants attempt to reduce applicator waste and improve convenience, but have not completely eliminated plastic applicators or wasteful packaging.

### SUMMARY

The present disclosure provides an applicator-free deodorant formed into a three dimensional body that is easy to transport and apply in a single step, as it spreads by body heat. Because it is applicator-free, there is no waste associated with the product.

In one embodiment, an applicator-free deodorant is made of a natural, plant-based composition formed into a three dimensional body that is easy to grasp and discretely place in the underarm in a single step where it melts by body heat into a liquid. The applicator-free deodorant is solid at room temperature, and melts in approximately 0 to 10 seconds after placement in the underarm, thereby coating the skin of the underarm and functioning as a deodorant.

In one embodiment, the applicator-free deodorant comprises a plant based oil and at least one absorbent powder. Other embodiments may optionally include sodium bicarbonate, an essential oil, or a wax, which is naturally occurring or derived from a plant-based source.

In one embodiment, the three dimensional shape is formed by heating the plant based oil and mixing at least one absorbent powder with the plant based oil until fully incorporated. In other embodiments, sodium bicarbonate, an essential oil, or a naturally occurring wax may optionally be incorporated into the mixture. The applicator-free deodorant is poured into a mold while in a liquid state and allowed to cool and solidify in order to form a three dimensional shape. In other embodiments, the three dimensional shape is formed by stamping or cutting shapes from a larger block, or by extruding the applicator-free deodorant from a reservoir.

The three dimensional body may be any of a variety of shapes including a cube. In other embodiments, the three dimensional body may be formed into the shape of a sphere, a heart shape, a cylinder, a diamond or any other shape that may be placed in the underarm in order to melt the deodorant through body heat by being sandwiched under a user's arm. In all embodiments, the three dimensional body is small enough to fit under the arm and be discretely applied, which can be beneficial as people travel more frequently, and work longer days.

In order to use the applicator-free deodorant, the user selects a three dimensional body and places it in the underarm. The user then lowers the arm in order to sandwich the applicator-free deodorant between the upper and lower arm inner surface of the underarm. When thus positioned, the outer surfaces of the solid, three dimensional body contact the underarm skin of the user on at least two inner surfaces, thus heating and melting the three dimensional body into a liquid, dispersing and coating the skin of the underarm to function as a deodorant.

### BRIEF DESCRIPTION OF THE DRAWINGS

Various aspects of at least one embodiment are discussed below with reference to the accompanying figures, which are not necessarily drawn to scale, emphasis instead being placed upon illustrating the principles disclosed herein. The figures are included to provide an illustration and a further understanding of the various aspects and embodiments, and are incorporated in and constitute a part of this specification, but are not intended as a definition of the limits of any particular embodiment. The figures, together with the remainder of the specification, serve to explain principles and operations of the described and claimed aspects and embodiments. In the figures, each identical or nearly identical component that is illustrated in various figures is represented by a like numeral. For purposes of clarity, not every component may be labeled in every figure.

FIG. 1 is a perspective view of the applicator-free deodorant composition according to a first embodiment of the present disclosure;

FIG. 2 is a cross sectional view of the applicator-free deodorant composition according to a first embodiment of the present disclosure;

FIG. 3 is a perspective view of the applicator-free deodorant composition as packaged according to a first embodiment of the present disclosure;

FIG. 4 is a perspective view of the applicator-free deodorant composition as packaged according to a first embodiment of the present disclosure;

FIG. 5 is a perspective view of the applicator-free deodorant composition in use according to a first embodiment of the present disclosure;

FIG. 6 is a perspective view of the applicator-free deodorant composition in use according to a first embodiment of the present disclosure;

FIG. 7 is a perspective view of the applicator-free deodorant in use according to a first embodiment of the present disclosure;

FIG. 8 is a perspective view of the applicator-free deodorant composition according to a second embodiment of the present disclosure;

FIG. 9 is a perspective view of the applicator-free deodorant composition according to a third embodiment of the present disclosure; and

FIG. 10 is a perspective view of the applicator-free deodorant composition according to a fourth embodiment of the present disclosure.

### DETAILED DESCRIPTION OF THE ILLUSTRATIVE EMBODIMENTS

The examples of the apparatus and method discussed herein are not limited in application to the details of construction and the arrangement of components set forth in the following description or illustrated in the accompanying drawings. It will be understood to one of skill in the art that the apparatus and methods are capable of implementation in other embodiments and of being practiced or carried out in various ways. Examples of specific embodiments are provided herein for illustrative purposes only and are not intended to be limiting. Also, the phraseology and terminology used herein is for the purpose of description and should not be regarded as limiting. Any references to examples, embodiments, components, elements or acts of the apparatus and method herein referred to in the singular may also embrace embodiments including a plurality, and any references in plural to any embodiment, component, element or act herein may also embrace embodiments including only a singularity. References in the singular or plural form are not intended to limit the presently disclosed apparatus or method, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms.

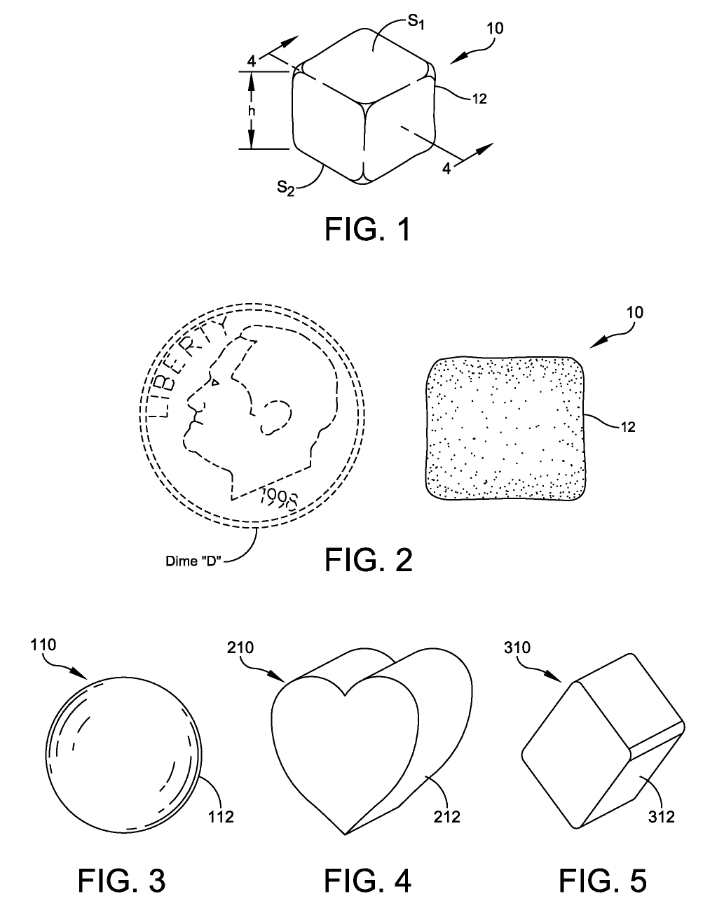

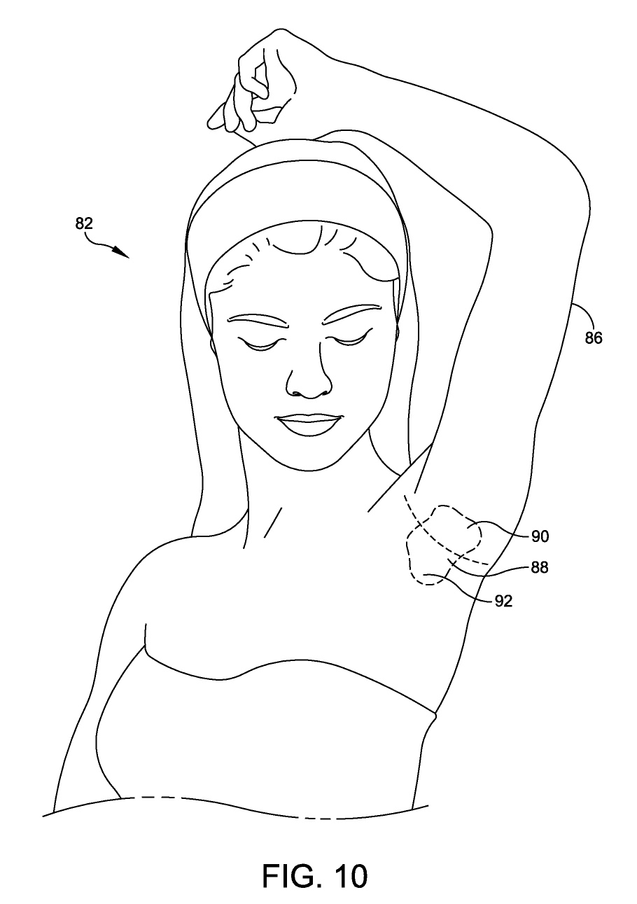

Referring initially to FIGS. 1-2, the applicator-free deodorant 10 is illustrated. The applicator-free deodorant 10 of the present embodiment includes a three dimensional body 12 that is solid and dimensionally stable at typical interior temperatures, and which melts into a liquid in a desired melting time at temperatures above approximately 90° F. to provide dispersion of the deodorant composition across the skin 88 when placed in underarm 84 of a user (FIG. 10). As used herein the term “applicator-free” means that dispersion of the deodorant onto the skin of the user is achieved without the use of an applicator used in conjunction with the three dimensional body 12, in order to support or hold the three dimensional body 12 as it is pressed against the skin 88 of the user 82. The three dimensional body 12 is sized to be easily grasped by the user 82 for placement in a crease of the underarm 84 formed by upper inner surface 90 and lower inner surface 92 of the underarm 84. In the present embodiment, three dimensional body 12 is cube-shaped and includes first and second outer surfaces (S₁ and S₂) that contact the upper inner surface 90 and the lower inner surface 92 of the underarm 84 so that the three dimensional body 12 melts by contact with the upper inner surface 90 and the lower inner surface 92 when the arm of the user is lowered, thereby dispersing the deodorant without the use of an applicator as described in greater detail below.

Because the deodorant composition is applicator-free, the size of three dimensional body 12 should be large enough to be easily grasped by the user 82 for placement in the crease of the underarm 84, while not exceeding the optimal volume of the applicator-free deodorant 10 needed to adequately cover the skin 88 of the underarm 84 of the user 82. The dime “D” of FIG. 2 provides a reference for the envisioning the approximate size of the three dimensional body 12, but the size of the three dimensional body 12 is not limited to the dimensions disclosed, as would be known to those of skill in the art.

The volume of the three dimensional body 12 is preferably large enough to provide for adequate coverage of the skin 88 of the upper inner surface 90 and the lower inner surface 92 of underarm 84 when melted, while avoiding the application of excess composition which may disperse beyond the desired area of the skin 88, or become deposited on the clothing when melted. In the present embodiment, the three dimensional body 12 is a cube with dimensions of approximately 1 cm on each side (S₁, S₂) with a volume of approximately 1 ml. The three dimensional body 12 may be larger than the present embodiment for use by individuals who are larger, who have additional body hair, or who otherwise desire coverage of a greater area of the skin 88, or sized smaller for use by smaller individuals or teens. The three dimensional body 12 of the present embodiment has a height “h” of approximately 0.65 cm to about 1.4 cm on each side, and the volume of the three dimensional body 12 may range from approximately 0.3 ml to about 2.5 ml. The applicator-free deodorant 10 is formulated to have a melting point in the range of from about 90° F. to about 99° F., preferably of about 92° F., and the proportions of the ingredients, are selected in order to produce a deodorant composition, which will remain solid and dimensionally stable at room temperature. The composition of the present embodiment includes at least one oil, preferably a plant-derived oil, and at least one absorbent powder. The composition may also optionally include essential oils, other natural fragrances, or sodium bicarbonate.

The at least one natural, plant-derived oil may include, but is not limited to, coconut oil, shea butter, cocoa butter, mango butter, avocado butter, or some combination thereof. Alternately, other plant-derived oils may be utilized, as would be known to those of skill in the art. The plant-derived oil provides the applicator-free deodorant 10 with a creamy feel when applied to the skin 88. Certain oils, such as coconut oil, also possess anti-bacterial and anti-microbial properties, which reduce the growth of bacteria, thereby reducing or eliminating odor. The plant-derived oil comprises approximately 33% of the composition, but in other embodiments the proportion of the plant-derived oil can range from approximately 22%-44% of the composition. As the oil melts after application to the crease of the underarm 84, it disperses the absorbent powder across the skin 88 and helps to adhere the absorbent powder to the skin 88.

The least one absorbent powder of the present composition may include, but is not limited to arrowroot powder, rice flour, tapioca flour, coconut flour, almond flour, ground charcoal, talc, cornstarch, or some combination thereof. Alternately, other absorbent powders may be utilized, as would be known to those of skill in the art. When dispersed across the skin 88 upon melting of the plant-based oil, the absorbent powder adheres to the skin 88 thereby absorbing perspiration and moisture. The absorbent powder also contributes to the dimensional stability of the applicator-free deodorant 10, and helps maintain the solid state of the applicator-free deodorant 10 when stored at room temperature. If desired, sodium bicarbonate may optionally be included in the composition in order to effectuate dimensional stability. Sodium bicarbonate also absorbs wetness and possesses antibacterial and antimicrobial properties, thereby reducing or eliminating odor.

In some embodiments, a mixture of absorbent powders may be used. By way of example, a mixture of equal parts arrowroot powder and baking soda may comprise the absorbent powder that is added to the plant-based oil in order to form the applicator-free deodorant 10. The absorbent powder comprises approximately 56% of the composition, but in other embodiments the proportion of the plant-derived oil can range from approximately 44%-67% of the composition.

Essential oils including, but not limited to, lavender oil, sweet orange oil, lemongrass oil, tea tree oil, eucalyptus oil, peppermint oil, bergamot oil, oregano oil, thyme oil, clove oil, cassia oil, geranium oil, manuka oil, and cypress oil may optionally be including in the composition. These oils are listed by way of example only, and any essential oil that is suitable for the skin 88 may be used in the composition as desired, as will be appreciated by those of skill in the art. If included in the applicator-free deodorant 10, the essential oil generally comprises approximately 5% of the composition, but in other embodiments the proportion of the essential oil can range from approximately 3%-11% of the composition, provided that dimensional stability is maintained.

In warmer climates, when the applicator-free deodorant 10 is stored in warmer conditions, or in other situations in which increased dimensional stability of the three dimensional body 12 is desired, a wax may optionally be included in the composition. The addition of candelilla wax or beeswax to the composition improves the dimensional stability of the three dimensional body without raising the melting point of the applicator-free deodorant 10. These waxes are listed by way of example only, and any wax that is naturally occurring or derived from a plant-based source, may be used in the composition as will be appreciated by those of skill in the art. If included in the applicator-free deodorant 10, the wax comprises approximately 6% of the composition, but in other embodiments the proportion of the wax can range from approximately 4%-12% of the composition.

The applicator-free deodorant 10 is formed into a solid three dimensional body 12 that is sized and shaped to be easily grasped by the user 82 for placement in the crease of the underarm 84. In order to create the three dimensional body 12, at least one plant-derived oil is heated until liquefied, and at least one absorbent powder is added and incorporated into the liquefied oil in proportions that produce a composition with the desired stability and melting point. In the present embodiment, the plant-derived oil is coconut oil, which comprises approximately 40% of the composition. The absorbent powder is seven parts arrowroot powder to one-part rice flour, which comprises approximately 60% of the composition. Additional ingredients, including an essential oil, a naturally occurring wax or sodium bicarbonate as described above, may be optionally included in the composition and mixed until fully incorporated throughout the liquid.

In another embodiment, the plant-derived oil is coconut oil which comprises approximately 33% of the composition, the absorbent powder is a one-to-one mixture of arrowroot powder and sodium bicarbonate which comprises approximately 56% of the composition, the essential oil is lavender or sweet orange oil which comprises approximately 5% of the composition, and the wax is candellila wax which comprises approximately 6% of the composition.

Once the desired composition is mixed, it is poured into a mold while in a liquid state where it cools and solidifies. By way of example, the mold may be a silicon mold. After the composition has solidified, the three dimensional body 12 is released from the mold and packaged for sale. Solidification may be accelerated by cooling, as would be known to those of skill in the art.

Although the three dimensional body 12 of the present embodiment is formed by molding, the three dimensional body 12 may be formed by other methods, such as cutting or stamping a three dimensional body 12 from a larger block of the applicator-free deodorant composition, or by extruding the partially cooled applicator-free deodorant composition from a reservoir. Alternately, other methods of forming the applicator-free deodorant composition into a three dimensional body 12 may be utilized, as would be known to those of skill in the art.

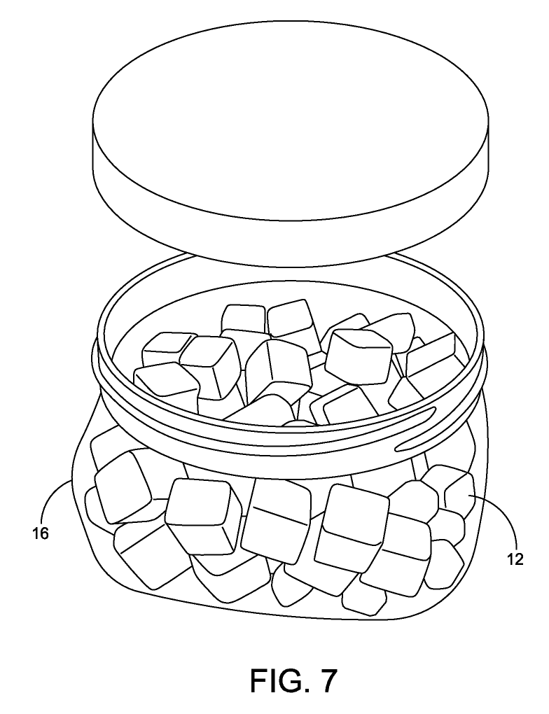

Referring now to FIG. 3, another embodiment of an applicator-free deodorant 110 is shown in the form of a sphere 112. In this embodiment, the same or similar elements of the embodiment of FIGS. 1-7 are labeled with the same reference numbers preceded with the numeral “1.” This embodiment is the same as the previous embodiment, except that the exemplary three dimensional body 112 is in the shape of a sphere. The three dimensional body 112 of the present embodiment has a height or diameter of approximately 0.65 cm to about 1.4 cm, and the volume of the three dimensional body 112 may range from approximately 0.3 ml to about 2.5 ml. Again, as described above, three dimensional body 112 is formed of an applicator-free deodorant 110 formulated to melt at approximately 92° F. Three dimensional body 112 is constructed and arranged to be easily grasped by the user 182, and to melt from instantaneously, namely less than about 0.1 seconds, up to about ten seconds upon placement in the crease of the underarm 184. In this embodiment, the sphere has a single, outer surface, where a first and second portion (S₁ and S₂) of the outer surface of the three dimensional body 112 may contact the upper inner surface 190 and the lower inner surface 192 of the skin 188. The three dimensional body 112 is sized to provide coverage of the skin 188 of the underarm 184 that is adequate for providing deodorant protection, as described above. The outer surface of the sphere 112 is free of any support or applicator as also described above. The shape of the three dimensional body is not limited to the shape of a sphere which is shown by way of example only.

Referring now to FIG. 4, another embodiment of an applicator-free deodorant 210 is shown in the form of a heart 212. In this embodiment, the same or similar elements of the embodiment of FIGS. 1-7 are labeled with the same reference numbers preceded with the numeral “2.” This embodiment is the same as the previous embodiment, except that the exemplary three dimensional body 212 is in the shape of a heart. The three dimensional body 212 of the present embodiment has a height of approximately 0.65 cm to about 1.4 cm, and the volume of the three dimensional body 212 may range from approximately 0.3 ml to about 2.5 ml. Again, as described above, three dimensional body 212 is formed of an applicator-free deodorant 210 formulated to melt at approximately 92° F. Three dimensional body 212 is constructed and arranged to be easily grasped by the user 282, and to melt from instantaneously to up to about ten seconds upon placement in the crease of the underarm 284 while providing coverage of the skin 288 of the underarm 284 that is adequate for providing deodorant protection, as described above. The outer surfaces of the heart shape 212 are free of any support or applicator as also described above in previous embodiments. The shape of the three dimensional body is not limited to the shape of a heart which is shown by way of example only.

Referring now to FIG. 5, another embodiment of an applicator-free deodorant 310 is shown in the form of a diamond 312. In this embodiment, the same or similar elements of the embodiment of FIGS. 1-7 are labeled with the same reference numbers preceded with the numeral “3.” This embodiment is the same as the previous embodiment, except that the exemplary three dimensional body 312 is in the shape of a diamond. As such, the three dimensional body 312 of the present embodiment has a height of approximately 0.65 cm to about 1.4 cm, and the volume of the three dimensional body 312 may range from approximately 0.3 ml to about 2.5 ml. Again, as described above, three dimensional body 312 is formed of an applicator-free deodorant 310 formulated to melt at approximately 92° F. Three dimensional body 312 is constructed and arranged to be easily grasped by the user 382, and to melt from instantaneously to up about ten seconds upon placement in the crease of the underarm 384 while providing coverage of the skin 388 of the underarm 384 that is adequate for providing deodorant protection, as described above. The outer surfaces of the diamond 312 are free of any support or applicator as also described above in previous embodiments. The shape of the three dimensional body is not limited to the shape of a diamond which is shown by way of example only.

Referring now to FIGS. 3-4, for any of the above embodiments, three dimensional bodies 12 may be optionally placed loosely in a container 16 for storage. In this embodiment, container 16 is a jar with a screw on lid, but any appropriately sized container 16 may be used, as will be appreciated by those of skill in the art. Container 16 is preferably made from a reusable or recyclable material, thereby reducing or eliminating the use of wasteful packaging. Three dimensional bodies 12 may easily be placed in a smaller container 16 for ease of transport, use, and convenience while traveling or at work.

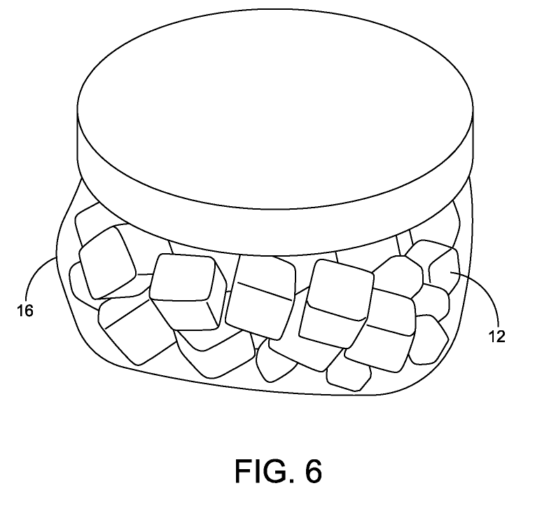

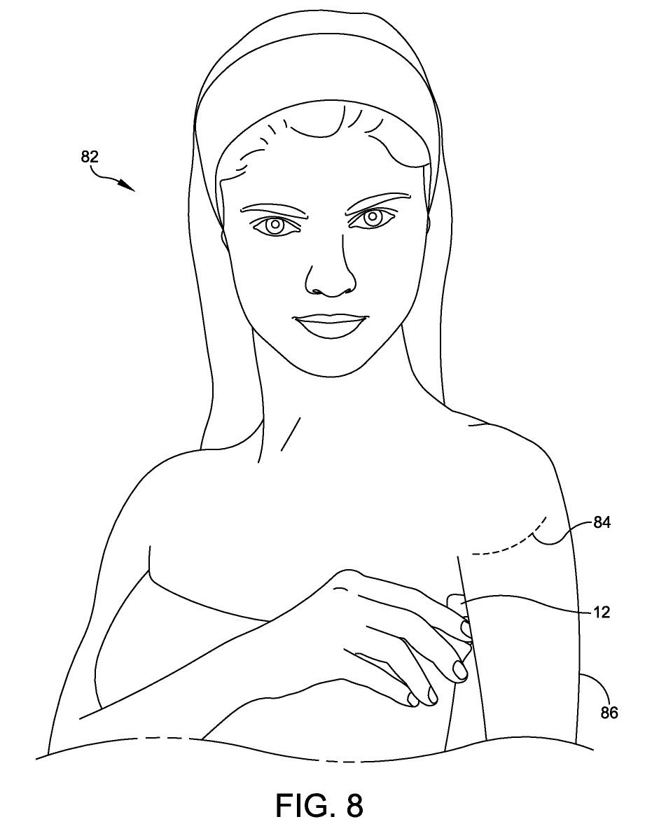

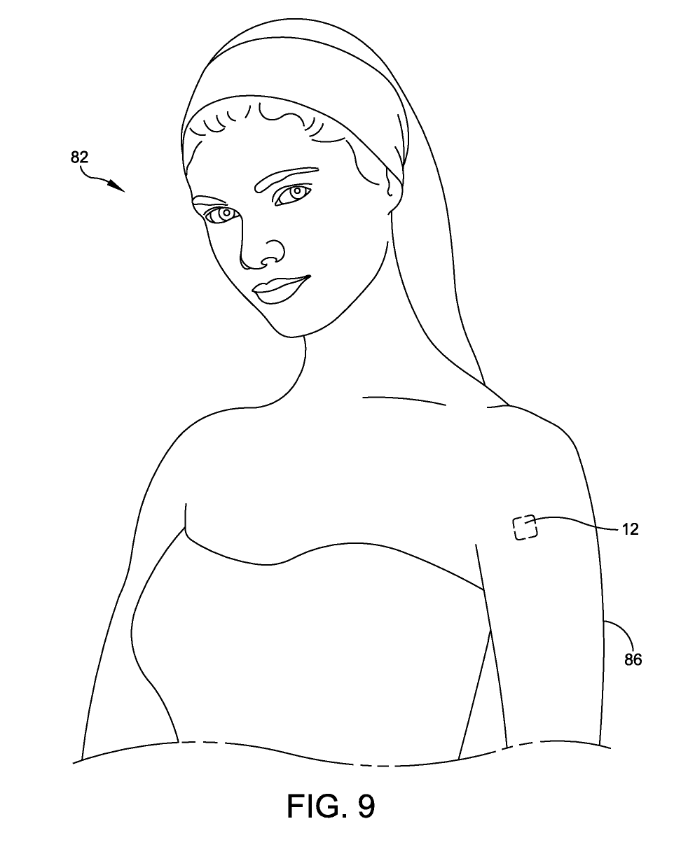

Use of the applicator-free deodorant will now be described with reference to FIGS. 6-10. In order to apply the deodorant composition, the user 82 selects a three dimensional body 12, and places the three dimensional body 12 into the crease of the underarm 84. Although no applicator is used to apply the applicator-free deodorant, the user my select a three dimensional body 12 by hand, by using tweezers, or by using any other implement suitable for grasping the three dimensional body 12 in order to place the three dimensional body 12 into the crease of the underarm 84. Once so placed, the user 82 lowers the arm 86, thereby sandwiching the three dimensional body 12 in between the upper inner surface 90 and lower inner surface 92 of the skin 88 of the underarm 84. As placed, at least outer surface S₁ of the three dimensional body 12 contacts with the upper inner surface 90, and at least outer surface S₂ of the three dimensional body 12 contacts with the lower inner surface 92 of the skin 88 of the underarm 84. Since the outer surfaces of the three dimensional body are free or any support or applicator, the upper inner surface 90 and lower arm inner surface 92 of the skin 88 of the underarm 84 are free to sandwich the three dimensional body 12. This contact of preferably at least two outer surfaces (S₁ and S₂) of the three dimensional body 12 with at both the upper arm inner surface 90 and lower arm inner surface 92 of the skin 88 facilitates the rapid warming, melting, and dispersion of the composition across the skin 88 of the underarm 84, thereby functioning as a deodorant for the user 82.

The composition is formulated to melt at approximately 92° F. Melting time and dispersion of the three dimensional body 10 will vary based on storage temperature of three dimensional body 12 and the body temperature of the user 82. When the applicator-free deodorant 10 is stored at a typical room temperature of approximately 69-72° F., melting and dispersion of the three dimensional body 12 after placement in the crease of the underarm 84 is almost instantaneous, occurring in less than about 0.1 seconds. Cooler storage temperatures or user 82 body temperatures increase the melting time of the three dimensional body 12, and under such circumstances, melting and dispersion of the three dimensional body 12 in the crease of the underarm 84 may take up to about ten seconds. Ideally, melting time is under 5 seconds.

In order to accelerate dispersion of the applicator-free deodorant 10 or to increase the area of the skin 88 covered by the applicator-free deodorant 10, the user 82 may move the arm while maintaining contact between the outer surfaces (S₁ and S₂) of the three dimensional body 12 and the upper arm inner surface 90 and lower arm inner surface 92 of the skin 88. Alternatively, the user 82 may disperse the applicator-free deodorant 10 in addition to or in place of sandwiching the three dimensional body 12 between the upper arm inner surface 90 and lower arm inner surface 92 of the skin 88, by rubbing the three dimensional body 12 directly on the skin 88 of the underarm 84 by hand. In this instance, the applicator-free deodorant 10 also melts by body heat, and dispersion of the applicator-free deodorant 10 is accelerated by rubbing. This method of application may be useful when the skin 88 of underarm 84 is not smooth due to hair growth. Alternatively, if increased coverage of the skin 88 is desired after melting of the three dimensional body 12 in the arm crease as described above is complete, the user 82 may spread the melted applicator-free deodorant 10 by hand, thus further distributing the applicator-free deodorant composition 10 across the skin 88. Individuals who desire coverage of a larger area of the skin 88 of the underarm 84 or who desire increased deodorant protection, may apply more than one three dimensional body 12 by any of the methods described above.

It will be understood by those skilled in the art that various changes in form and details may be made without departing from the spirit and scope of the invention as defined by the appended claims. For example, the materials disclosed herein may be readily changed, as may the dimensions and geometric configurations of the components described herein, i.e. the applicator-free deodorant can have other shapes and sizes, and melting time once placed in the underarm may vary. Therefore, the above description should not be construed as limiting, but merely as exemplifications of preferred embodiments. Those skilled in the art will envision other modifications within the scope, spirit and intent of the invention.

What is claimed is:

1. An applicator-free deodorant, for use in an underarm having an upper inner surface and a lower inner surface, the applicator-free deodorant comprising:
a three dimensional body having one or more outer surfaces, each of the one or more outer surfaces being free of an external applicator support, the three dimensional body being constructed and arranged such that at least one of the outer surfaces of the three dimensional body contacts the upper inner surface and at least one of the outer surfaces contacts the lower inner upon placement in the underarm, the three dimensional body further having a composition including at least a plant-derived oil and an absorbent powder; and
wherein the three dimensional body is a dimensionally stable solid at temperatures of up to about 90° F., and melts into a liquid by the user's body temperature in about 0.01 seconds to about 10 seconds upon placement in the underarm, thereby dispersing the deodorant composition across portions of both the upper inner surface and the lower inner surface of the underarm.

2. The applicator-free deodorant of claim 1, wherein the shape of the three dimensional body is selected from the group consisting of a cube, a sphere, a heart, and a diamond.

3. The applicator-free deodorant of claim 1, wherein the three dimensional body has a height of about 0.65 cm to about 1.0 cm.

4. The applicator-free deodorant of claim 1, wherein the three dimensional body is about 0.3 ml to about 2.5 ml in volume.

5. The applicator-free deodorant of claim 1, wherein the melting point of the three dimensional body is about 90° F. to about 99° F.

6. The applicator-free deodorant of claim 1, wherein the plant-derived oil is selected from a group consisting of a coconut oil, a shea butter, a cocoa butter, a mango butter, an avocado butter, and any combination thereof.

7. The applicator-free deodorant of claim 6, wherein the absorbent powder is selected from a group consisting of an arrowroot powder, a rice flour, a tapioca flour, a coconut flour, an almond flour, a ground charcoal, talc, cornstarch, and any combination thereof.

8. The applicator-free deodorant of claim 7, wherein a volume of the three dimensional body is comprised of about 22% to about 44% of the plant-derived oil and about 44% to about 66% of the absorbent powder.

9. The applicator-free deodorant of claim 8, wherein the absorbent powder is comprised of about 50% arrowroot powder and about 50% sodium bicarbonate.

10. The applicator-free deodorant of claim 8, wherein the absorbent powder is comprised of about 12.5% rice flour and about 87.5% arrowroot powder.

11. The applicator-free deodorant of claim 8, wherein the composition of the three dimensional body further comprises about 3% to about 11% of an essential oil.

12. The applicator-free deodorant of claim 8, wherein the composition of the three dimensional body further comprises about 4% to about 12% of a wax.

13. An applicator-free deodorant, for use in an underarm having an upper inner surface and a lower inner surface, the applicator-free deodorant comprising:
a three dimensional body having:
a) one or more outer surfaces, each one or more of the outer surfaces being free of an external applicator support, the three dimensional body being constructed and arranged such that at least one of the outer surfaces of the three dimensional body contacts the upper inner surface and at least one of the outer surfaces contacts the lower inner upon placement in the underarm;
b) a volume of about 0.3 ml to about 2.5 ml;
c) a composition comprising at least about 22% to about 44% plant-derived oil, about 44% to about 67% absorbent powder, and about 4% to about 12% wax of the volume; and
wherein the three dimensional body is a dimensionally stable solid at temperatures of up to about 90° F., and melts into a liquid by the user's body temperature in about 0.01 seconds to about 10 seconds upon placement in the underarm, thereby dispersing the deodorant composition across portions of both the upper inner surface and the lower inner surface of the underarm.

14. The applicator-free deodorant of claim 13, wherein the plant-derived oil is selected from a group consisting of an arrowroot powder, a rice flour, a tapioca flour, a coconut flour, an almond flour, a ground charcoal, talc, cornstarch, and any combination thereof.

15. The applicator-free deodorant of claim 13, wherein the absorbent powder is selected from a group consisting of an arrowroot powder, a rice flour, a tapioca flour, a coconut flour, an almond flour, a ground charcoal, talc, cornstarch, and any combination thereof.

16. The applicator-free deodorant of claim 15, wherein the absorbent powder comprises about 12.5% rice flour and about 87.5% arrowroot powder.

17. The applicator-free deodorant of claim 13, further comprising about 3% to about 11% of an essential oil.

18. The applicator-free deodorant of claim 13, wherein the wax is selected from the group consisting of candellila wax and beeswax.

19. The applicator-free deodorant of claim 13, wherein the shape of the solid three dimensional body is selected from the group consisting of a cube, a sphere, a heart, and a diamond.

20. A method of making an applicator-free deodorant for use in an underarm having an upper inner surface and a lower inner surface, the method comprising:
heating at least one plant-derived oil until liquefied;
mixing the at least one plant-derived oil with at least one absorbent powder to form a deodorant composition, the at least one plant-derived oil comprising about 22% to about 44% of the volume of the composition, the at least one absorbent powder comprising about 44% to about 67% of the deodorant composition; and
forming the deodorant composition into a solid three dimensional body including one or more outer surfaces, each outer surface being constructed and arranged to contact the upper inner surface and the lower inner surface upon placement of the three dimensional body in the underarm, the three dimensional body being dimensionally stable at temperatures of up to about 90° F., and melting in to a liquid through body temperature in about 0.01 seconds to about 10 seconds upon placement in the underarm, in order to disperse the deodorant composition to the underarm.

21. The method of claim 20, wherein forming the three dimensional body further comprises the steps of pouring the liquefied deodorant composition into a mold, cooling the liquefied deodorant until the solid three dimensional body is formed, and removing the solid three dimensional body having a volume of about 0.3 ml to about 2.5 ml from the mold.
